SEQUENCE LISTING (2 sequences)

SEQ ID NO: 1 - protein, 458 aa, Homo sapiens
MLPDFMLVLIVLGIPSSATTGFNSIAENEDALLRHLFQGYQKWVRPVLHSNDTIKVYFGLKISQLVDVDEKNQLMTTNVWLKQEWTDHKLRWNPDDYGGIHSIKVPSESLWLPDIVLFENADGRFEGSLMTKVIVKSNGTVVWTPPASYKSSCTMDVTFFPFDRQNCSMKFGSWTYDGTMVDLILINENVDRKDFFDNGEWEILNAKGMKGNRRDGVYSYPFITYSFVLRRLPLFYTLFLIIPCLGLSFLTVLVFYLPSDEGEKLSLSTSVLVSLTVFLLVIEEIIPSSSKVIPLIGEYLLFIMIFVTLSIIVTVFVINVHHRSSSTYHPMAPWVKRLFLQKLPKLLCMKDHVDRYSSPEKEESQPVVKGKVLEKKKQKQLSDGEKVLVAFLEKAADSIRYISRHVKKEHFISQVVQDWKFVAQVLDRIFLWLFLIVSVTGSVLIFTPALKMWLHSYH

SEQ ID NO: 2 - DNA, 2347 bp, Homo sapiens
  source 1..2347: mol_type = other DNA
gagaagcggcacactcggcgagaggggttgagattgttttattccactccaggtgttgctgtcctcttgggttccacttcggattttgaacccctgtattttcttttcaaaacccccttttccagtggaaatgctctgttgttaaaaaggaagaaactgtctttctgaaactgacatcacgatgctcccagattttatgctggttctcatcgtccttggcatcccttcctcagccaccacaggtttcaactcaatcgccgaaaatgaagatgccctcctcagacatttgttccaaggttatcagaaatgggtccgccctgtattacattctaatgacaccataaaagtatattttggattgaaaatatcccagcttgtagatgtggatgaaaagaatcagctgatgacaaccaatgtgtggctcaaacaggaatggacagaccacaagttacgctggaatcctgatgattatggtgggatccattccattaaagttccatcagaatctctgtggcttcctgacatagttctctttgaaaatgctgacggccgcttcgaaggctccctgatgaccaaggtcatcgtgaaatcaaacggaactgttgtctggacccctcccgccagctacaaaagctcctgcaccatggacgtcacgtttttcccgttcgaccgacagaactgctccatgaagtttggatcctggacttatgatggcaccatggttgacctcattttgatcaatgaaaatgtcgacagaaaagacttcttcgataacggagaatgggaaatactgaacgcaaaggggatgaaggggaacagaagggacggcgtgtactcctatccctttatcacgtattccttcgtcctgagacgcctgcctttattctataccctctttctcatcatcccctgcctggggctgtctttcctaacagttcttgtgttctatttaccttcggatgaaggagaaaaactttcattatccacatcggtcttggtttctctgacagttttccttttagtgattgaagaaatcatcccatcgtcttccaaagtcattcctctcattggagagtacctgctgttcatcatgatttttgtgaccctgtccatcattgttaccgtgtttgtcattaacgttcaccacagatcttcttccacgtaccaccccatggccccctgggttaagaggctctttctgcagaaacttccaaaattactttgcatgaaagatcatgtggatcgctactcatccccagagaaagaggagagtcaaccagtagtgaaaggcaaagtcctcgaaaaaaagaaacagaaacagcttagtgatggagaaaaagttctagttgcttttttggaaaaagctgctgattccattagatacatttcgagacatgtgaagaaagaacattttatcagccaggtagtacaagactggaaatttgtagctcaagttcttgaccgaatcttcctgtggctctttctgatagtgtcagtaacaggctcggttctgatttttacccctgctttgaagatgtggctacatagttaccattaggaatttaaaagacataagactaaattacaccttagacctgacatctggctatcacacagacagaatccaaatgcatgtgcttgttctacgaaccccgaatgcgttgtctttgtggaaatggaacatctcctcatgggagaaactctggtaaatgtgctcatttgtggttgccatgagagtgagctgcttttaaagaaagtggagcctcctcagacccctgccttggctttcccagacattcagggagggatcataggtccaggcttgagctcacatgtggccagagtgcacaaaaagctgttgctacttggtggaggaacacctcctagaagcagcaggcctcggtggtgggggaggggggattcacctggaattaaggaagtctcggtgtcgagctatctgtgtgggcagagcctggatctcccaccctgcactggcctccttggtggttttgtctgagctgcaaactggggccgtgttatgcttgttagcaatgggccaggcaaatctcaacaagggtttggggattacactggcaggatagctggcactgcacacttaacaataacacagattccaagttgcctggcctaccgaagcaggtgttattcatggtttattcccagcatgataaggctttcagatggtgaaactggagcccactgtgagctgtgacttcctttaaggtcatgcataccaaaataactcacaatagaaacaaagccctttgctaccagaagtggattcattaatacccattatcttttcctagtaaactagtgtacagaa

(19) United States
(12) Patent Application Publication
(10) Pub. No.: US 2025/0136996 A1
(43) Pub. Date: May 1, 2025

(54) TREATMENT OF SMOKING WITH CHOLINERGIC RECEPTOR NICOTINIC BETA 3 SUBUNIT (CHRNB3) INHIBITORS

(71) Applicant: Regeneron Pharmaceuticals, Inc., Tarrytown, NY (US)

(72) Inventors: Veera Rajagopal, White Plains, NY (US); Giovanni Coppola, Chappaqua, NY (US); Aris Baras, Greenwich, CT (US); Jonathan Marchini, Oxfordshire (GB); Andrey Ziyatdinov, Ridgewood, NJ (US); Mohsin Ahmed, West Harrison, NY (US); Olivier Harari, New York, NY (US)

(21) Appl. No.: 18/809,138

(22) Filed: Aug. 19, 2024

Related U.S. Application Data
(60) Provisional application No. 63/593,164, filed on Oct. 25, 2023.

(51) Int. Cl. C12N 15/113; A61K 31/7088; A61K 38/46; A61P 25/34

(52) U.S. Cl. CPC C12N 15/1138; A61K 31/7088; A61K 38/465; A61P 25/34; C12N 2310/11; C12N 2310/14; C12N 2310/531

(57) ABSTRACT

The present disclosure provides methods of treating subjects having a smoking addiction/disorder, and methods of identifying subjects having an increased risk of developing a smoking addiction/disorder.

### PRIORITY

This application claims the benefit of priority of the filing date of U.S. provisional application No. 63/593,164, filed on Oct. 25, 2023, the disclosure of which is incorporated by reference herein in its entirety.

### INCORPORATION BY REFERENCE OF SEQUENCE LISTING

This application contains a Sequence Listing which has been submitted electronically in ST26 format and hereby incorporated by reference in its entirety. Said ST26 file, created on Aug. 7, 2024, is name 6341002US11.xml and is 5,676 bytes in size.

### FIELD

The present disclosure relates generally to the treatment of subjects for smoking with cholinergic receptor nicotinic beta 3 subunit (Neuronal acetylcholine receptor subunit beta-3; CHRNB3) inhibitors, and methods of identifying subjects having an increased risk of developing a smoking habit.

### SUMMARY

The present disclosure provides methods of treating a subject for smoking or at risk of developing a smoking habit, the methods comprising administering an CHRNB3 inhibitor to the subject.

One aspect provides a method to reduce smoking in a subject, the method comprising administering an inhibitor of cholinergic receptor nicotinic beta 3 subunit (CHRNB3) to the subject. In one aspect, the subject smokes 1-2 fewer cigarettes per day as compared to smoking prior to treatment. In another aspect, the subject smokes 3-4 fewer cigarettes per day as compared to smoking prior to treatment. In one aspect, the subject smokes 4-5 fewer cigarettes per day as compared to smoking prior to treatment. In one aspect, the subject smokes 4-6 fewer cigarettes per day as compared to smoking prior to treatment. In one aspect, the subject smokes 7 or more fewer cigarettes per day as compared to smoking prior to treatment.

In one aspect, the inhibitor of CHRNB3 comprises a small molecule.

In one aspect, the inhibitor or CHRNB3 comprises an inhibitory nucleic acid molecule, such as an antisense nucleic acid molecule, a small interfering RNA (siRNA), a short hairpin RNA (shRNA) or a combination thereof that hybridizes to a CHRNB3 mRNA or DNA.

In one aspect, the inhibitor of CHRNB3 comprises a Cas protein and guide RNA (gRNA) that hybridizes to a gRNA recognition sequence within a CHRNB3 genomic nucleic acid molecule.

In one aspect, the method further comprises detecting the presence or absence of an CHRNB3 variant nucleic acid molecule and/or a CHRNB3 predicted loss-of-function polypeptide or missense polypeptide in a biological sample from the subject. In one aspect, the detecting step is carried out in vitro. In one aspect, the detecting step comprises: obtaining or having obtained a biological sample from the subject; and performing or having performed an assay on the biological sample to determine whether the subject has the CHRNB3 variant nucleic acid molecule, and/or a CHRNB3 predicted loss-of-function polypeptide or missense polypeptide. In one aspect, the subject has an absence of an CHRNB3 variant nucleic acid molecule and/or a CHRNB3 predicted loss-of-function polypeptide and is administered the CHRNB3 inhibitor. In one aspect, the subject is heterozygous for a CHRNB3 variant nucleic acid molecule, and the subject is administered the CHRNB3 inhibitor.

One aspect provides a method of treating a subject with a cholinergic receptor nicotinic beta 3 subunit (CHRNB3) inhibitor, wherein the subject is need of reducing smoking or at risk of increasing smoking, the method comprising the steps of: determining whether the subject has a CHRNB3 variant nucleic acid molecule by: obtaining or having obtained a biological sample from the subject; and performing or having performed a sequence analysis on the biological sample to determine if the subject has a genotype comprising the CHRNB3 variant nucleic acid molecule; and administering or continuing to administer the CHRNB3 inhibitor in a standard dosage amount to a CHRNB3 reference subject; and administering or continuing to administer the CHRNB3 inhibitor in a dosage amount that is the same as or less than a standard dosage amount to a subject that is heterozygous for the CHRNB3 variant nucleic acid molecule; wherein the presence of a genotype having the CHRNB3 variant nucleic acid molecule indicates the subject has a decreased risk of increase in smoking/addiction.

### BRIEF DESCRIPTION OF THE DRAWINGS

FIG. 1 provides a Manhattan plot demonstrating, for example, associate statistics.

FIG. 2. The plot shows the association of rare variants in the genomic region around CHRNB3 in chromosome 8. The missense variant is labeled.

FIG. 3. The graph shows the distribution of cig-per-day in the three genotype groups of the Glu284Gly missense variant.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by +10% and remain within the scope of the disclosed embodiments.

As used herein, the term “comprising” may be replaced with “consisting” or “consisting essentially of” in particular embodiments as desired.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates (such as, for example, apes and monkeys). In some embodiments, the subject is a human. In some embodiments, the subject is a patient under the care of a physician.

It has been observed in accordance with the present disclosure that a gene burden of particular CHRNB3 variations (i.e., CHRNB3 variant nucleic acid molecules) associate with a decreased risk of developing s smoking disorder. It is believed that variants in CHRNB3 genes or proteins have not been significantly associated with smoking in previous exome-sequencing association studies. Therefore, it is believed that humans having a smoking disorder or at risk of developing a smoking disorder may be treated with CHRNB3 inhibitors. Accordingly, the present disclosure provides methods for leveraging the identification of subjects who do not have such protective CHRNB3 variant nucleic acid molecules who, thus, are at risk of developing a smoking disorder, and to stratify the risk in such subjects of developing smoking disorder, such that subjects at risk or subjects with active disease may be treated with CHRNB3 inhibitors.

In any of the embodiments described herein, the CHRNB3 variant nucleic acid molecules can be any CHRNB3 nucleic acid molecule (such as, for example, genomic nucleic acid molecule (e.g., human cholinergic receptor nicotinic beta 3 subunit (CHRNB3), Entrez 1142, Ensembl ENSG00000147432, the sequences of which are incorporated herein by reference), mRNA molecule (NM_000749, NM_001347717; protein sequence NP_000740, NP_001334646, the sequence of the mRNA and protein are each incorporated herein by reference), or cDNA molecule) encoding a CHRNB3 polypeptide having a partial loss-of-function, a complete loss-of-function, a predicted partial loss-of-function, or a predicted complete loss-of-function, or encoding a missense polypeptide, or resulting in the absence of an encoded polypeptide, or having an impact on the CHRNB3 mRNA sequence or expression. For example, the CHRNB3 variant nucleic acid molecules can be any of the CHRNB3 variant nucleic acid molecules described herein. A CHRNB3 variant nucleic acid molecule can be a variant that is predicted to result in the premature truncation of the CHRNB3 polypeptide (including, but not limited to, frameshift mutations, insertions or deletions, stop-gain, stop-lost, start-lost, splice site variants or large chromosomal or sub-chromosomal re-arrangements affecting the CHRNB3 gene). CHRNB3 variant nucleic acid molecules can include, but are not limited, to in-frame insertions or deletions in the CHRNB3 gene or variants in the untranslated regions of the CHRNB3 gene. A missense variant is a variant predicted to result in the change of an amino acid sequence of the CHRNB3 polypeptide.

For purposes of the present disclosure, any particular subject, such as a human, can be categorized as having one of three CHRNB3 genotypes: i) CHRNB3 reference; ii) heterozygous for a CHRNB3 variant nucleic acid molecule, and iii) homozygous for a CHRNB3 variant nucleic acid molecule. A subject is CHRNB3 reference when the subject does not have a copy of a CHRNB3 variant nucleic acid molecule. A subject is heterozygous for a CHRNB3 variant nucleic acid molecule when the subject has a single copy of a CHRNB3 variant nucleic acid molecule. A CHRNB3 variant nucleic acid molecule is any CHRNB3 nucleic acid molecule (such as, a genomic nucleic acid molecule, an mRNA molecule, or a cDNA molecule) encoding a CHRNB3 polypeptide having a partial loss-of-function, a complete loss-of-function, a predicted partial loss-of-function, or a predicted complete loss-of-function, or encoding a missense polypeptide, or having an impact on the CHRNB3 mRNA sequence. A subject who has a CHRNB3 polypeptide having a partial loss-of-function (or predicted partial loss-of-function, or a missense) is hypomorphic for CHRNB3 (lower abundance or function of the gene compared to the reference sequence version). The CHRNB3 variant nucleic acid molecule can be any variant nucleic acid molecule described herein. A subject is homozygous for a CHRNB3 variant nucleic acid molecule when the subject has two copies of any of the CHRNB3 variant nucleic acid molecules.

For subjects that are genotyped or determined to be heterozygous or homozygous for a CHRNB3 variant nucleic acid molecule, such subjects have a decreased risk of developing a smoking addiction/disorder compared to CHRNB3 reference subjects. For subjects that are genotyped or determined to be CHRNB3 reference, such subjects have an increased risk of developing a smoking addiction/disorder compared to carriers of the aforementioned CHRNB3 variants. For subjects that are genotyped or determined to be CHRNB3 reference or are heterozygous for a CHRNB3 variant nucleic acid molecule, such subjects can be treated with one or more CHRNB3 inhibitors. Such subjects can also be treated with therapeutic agents used to treat a smoking addiction/disorder.

In any of the embodiments described herein, the CHRNB3 variant nucleic acid molecule can be any nucleic acid molecule (such as, for example, genomic nucleic acid molecule, mRNA molecule, or cDNA molecule) encoding a CHRNB3 polypeptide having a partial loss-of-function, a complete loss-of-function, a predicted partial loss-of-function, or a predicted complete loss-of-function, or encoding a missense polypeptide, or having an impact on the CHRNB3 mRNA sequence. In some embodiments, the CHRNB3 variant nucleic acid molecule is a variant that causes or is predicted to cause a nonsynonymous amino acid substitution in CHRNB3 and whose allele frequency is less than 1/100 alleles in the population from which the subject is selected. In some embodiments, the CHRNB3 variant nucleic acid molecule is any rare missense variant (allele frequency <1%; or 1 in 100 alleles) or any missense variant predicted or demonstrated to result in a change in CHRNB3 polypeptide sequence, folding, structure, abundance or function regardless of frequency, or any splice-site, stop-gain, start-loss, stop-loss, frameshift, or in-frame indel, or other frameshift CHRNB3 variant or any other variant predicted or demonstrated to result in a change in the amino acid sequence of the CHRNB3 polypeptide.

In any of the embodiments described herein, the CHRNB3 variant nucleic acid molecules have one or more variations of chromosome 8 using the nucleotide sequence of the CHRNB3 reference genomic nucleic acid molecule (ENSG00000147432 GRCh38.p14 (GCF_000001405.40); Chr 8 (NC_000008.11 (42697366 . . . 42737407)); Q05901 (protein)).

```
Human protein:
(SEQ ID NO: 1)
MLPDFMLVLIVLGIPSSATTGENSIAENEDALLRHLFQGYQKWVRPVLHSNDTIKVYFGLKISQLVDVDE
KNQLMTTNVWLKQEWTDHKLRWNPDDYGGIHSIKVPSESLWLPDIVLFENADGRFEGSLMTKVIVKSNGT
VVWTPPASYKSSCTMDVTFFPFDRQNCSMKFGSWTYDGTMVDLILINENVDRKDFFDNGEWEILNAKGMK
GNRRDGVYSYPFITYSFVLRRLPLFYTLFLIIPCLGLSFLTVLVFYLPSDEGEKLSLSTSVLVSLTVFLL
VIEEIIPSSSKVIPLIGEYLLFIMIFVTLSIIVTVFVINVHHRSSSTYHPMAPWVKRLFLQKLPKLLCMK
DHVDRYSSPEKEESQPVVKGKVLEKKKQKQLSDGEKVLVAFLEKAADSIRYISRHVKKEHFISQVVQDWK
FVAQVLDRIFLWLFLIVSVTGSVLIFTPALKMWLHSYH
Human mRNA:
(SEQ ID NO: 2)
      1 gagaagcggc acactcggcg agaggggttg agattgtttt attccactcc aggtgttgct
    61 gtcctcttgg gttccacttc ggattttgaa cccctgtatt ttcttttcaa aacccccttt
  121 tccagtggaa atgctctgtt gttaaaaagg aagaaactgt ctttctgaaa ctgacatcac
  181 gatgctccca gattttatgc tggttctcat cgtccttggc atcccttcct cagccaccac
  241 aggtttcaac tcaatcgccg aaaatgaaga tgccctcctc agacatttgt tccaaggtta
  301 tcagaaatgg gtccgccctg tattacattc taatgacacc ataaaagtat attttggatt
  361 gaaaatatcc cagcttgtag atgtggatga aaagaatcag ctgatgacaa ccaatgtgtg
  421 gctcaaacag gaatggacag accacaagtt acgctggaat cctgatgatt atggtgggat
  481 ccattccatt aaagttccat cagaatctct gtggcttcct gacatagttc tctttgaaaa
  541 tgctgacggc cgcttcgaag gctccctgat gaccaaggtc atcgtgaaat caaacggaac
  601 tgttgtctgg acccctcccg ccagctacaa aagctcctgc accatggacg tcacgttttt
  661 cccgttcgac cgacagaact gctccatgaa gtttggatcc tggacttatg atggcaccat
  721 ggttgacctc attttgatca atgaaaatgt cgacagaaaa gacttcttcg ataacggaga
  781 atgggaaata ctgaacgcaa aggggatgaa ggggaacaga agggacggcg tgtactccta
  841 tccctttatc acgtattcct tcgtcctgag acgcctgcct ttattctata ccctctttct
  901 catcatcccc tgcctggggc tgtctttcct aacagttctt gtgttctatt taccttcgga
  961 tgaaggagaa aaactttcat tatccacatc ggtcttggtt tctctgacag ttttcctttt
1021 agtgattgaa gaaatcatcc catcgtcttc caaagtcatt cctctcattg gagagtacct
1081 gctgttcatc atgatttttg tgaccctgtc catcattgtt accgtgtttg tcattaacgt
1141 tcaccacaga tcttcttcca cgtaccaccc catggccccc tgggttaaga ggctctttct
1201 gcagaaactt ccaaaattac tttgcatgaa agatcatgtg gatcgctact catccccaga
1261 gaaagaggag agtcaaccag tagtgaaagg caaagtcctc gaaaaaaaga aacagaaaca
1321 gcttagtgat ggagaaaaag ttctagttgc ttttttggaa aaagctgctg attccattag
1381 atacatttcg agacatgtga agaaagaaca ttttatcagc caggtagtac aagactggaa
1441 atttgtagct caagttcttg accgaatctt cctgtggctc tttctgatag tgtcagtaac
1501 aggctcggtt ctgattttta cccctgcttt gaagatgtgg ctacatagtt accattagga
1561 atttaaaaga cataagacta aattacacct tagacctgac atctggctat cacacagaca
1621 gaatccaaat gcatgtgctt gttctacgaa ccccgaatgc gttgtctttg tggaaatgga
1681 acatctcctc atgggagaaa ctctggtaaa tgtgctcatt tgtggttgcc atgagagtga
1741 gctgctttta aagaaagtgg agcctcctca gacccctgcc ttggctttcc cagacattca
1801 gggagggatc ataggtccag gcttgagctc acatgtggcc agagtgcaca aaaagctgtt
1861 gctacttggt ggaggaacac ctcctagaag cagcaggcct cggtggtggg ggagggggga
1921 ttcacctgga attaaggaag tctcggtgtc gagctatctg tgtgggcaga gcctggatct
1981 cccaccctgc actggcctcc ttggtggttt tgtctgagct gcaaactggg gccgtgttat
2041 gcttgttagc aatgggccag gcaaatctca acaagggttt ggggattaca ctggcaggat
2101 agctggcact gcacacttaa caataacaca gattccaagt tgcctggcct accgaagcag
2161 gtgttattca tggtttattc ccagcatgat aaggctttca gatggtgaaa ctggagccca
2221 ctgtgagctg tgacttcctt taaggtcatg cataccaaaa taactcacaa tagaaacaaa
2281 gccctttgct accagaagtg gattcattaa tacccattat cttttcctag taaactagtg
2341 tacagaa
```

In any of the embodiments described herein, the CHRNB3 variant nucleic acid molecules can be mRNA and cDNA molecules having the corresponding variant positions referring to the reference genomic sequence as a reference sequence.

The nucleotide and amino acid sequences provided are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence. In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned. A variety of computational algorithms exist that can be used for performing a sequence alignment to identify a nucleotide or amino acid position in one polymeric molecule that corresponds to a nucleotide or amino acid position in another polymeric molecule. For example, by using the NCBI BLAST algorithm (Altschul et al., Nucleic Acids Res., 1997, 25, 3389-3402) or CLUSTALW software (Sievers and Higgins, Methods Mol. Biol., 2014, 1079, 105-116) sequence alignments may be performed. However, sequences can also be aligned manually.

Any one or more (i.e., any combination) of the variants recited herein can be used within any of the methods described herein to determine whether a subject has an increased or decreased risk of developing a smoking addiction/disorder. The combinations of particular variants can form a gene-burden or “mask” used for statistical analysis of the particular correlation of CHRNB3 and higher or lower risk of developing a smoking addiction/disorder.

In any of the embodiments described herein, the CHRNB3 predicted loss-of-function polypeptide can be any CHRNB3 polypeptide having a partial loss-of-function, a complete loss-of-function, a predicted partial loss-of-function, or a predicted complete loss-of-function, or missense polypeptide.

In some embodiments, the CHRNB33 inhibitor comprises an inhibitory nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of a CHRNB3 mRNA. In some embodiments, the antisense RNA, siRNA, or shRNA hybridizes to a sequence within a CHRNB3 genomic nucleic acid molecule or mRNA molecule and decreases expression of the CHRNB3 polypeptide in a cell in the subject. In some embodiments, the CHRNB3 inhibitor comprises an antisense RNA that hybridizes to a CHRNB3 genomic nucleic acid molecule or mRNA molecule and decreases expression of the CHRNB3 polypeptide in a cell in the subject. In some embodiments, the CHRNB3 inhibitor comprises an siRNA that hybridizes to a CHRNB3 genomic nucleic acid molecule or mRNA molecule and decreases expression of the CHRNB3 polypeptide in a cell in the subject. In some embodiments, the CHRNB3 inhibitor comprises an shRNA that hybridizes to a CHRNB3 genomic nucleic acid molecule or mRNA molecule and decreases expression of the CHRNB3 polypeptide in a cell in the subject. The inhibitory nucleic acid molecules described herein can be targeted to various CHRNB3 transcripts.

The inhibitory nucleic acid molecules disclosed herein can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules disclosed herein can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The inhibitory nucleic acid molecules disclosed herein can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules disclosed herein can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH2 and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N6-methyladenosine, inosine, and N7-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula:

Sense: mN*mN*/i2FN/mN/i2FN/mN/i2FN/mN/i2FN/mN/i2FN/mN/i2FN/mN/i2FN/mN/i2FN/*mN*/32FN/

Antisense: /52FN/*/12FN/*mN/i2FN/mN/i2FN/mN/i2FN/mN/i2FN/mN/i2FN/mN/i2FN/mN/i2FN/mN/12FN/mN*N*N
wherein: “N” is the base; “2F” is a 2′-F modification; “m” is a 2′-O-methyl modification, “I” is an internal base; and “*” is a phosphorothioate backbone linkage.

The present disclosure also provides vectors comprising any one or more of the inhibitory nucleic acid molecules disclosed herein. In some embodiments, the vectors comprise any one or more of the inhibitory nucleic acid molecules disclosed herein and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules disclosed herein. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, the CHRNB3 inhibitor comprises a nuclease agent that induces one or more nicks or double-strand breaks at a recognition sequence(s) or a DNA-binding protein that binds to a recognition sequence within a CHRNB3 genomic nucleic acid molecule. The recognition sequence can be located within a coding region of the CHRNB3 gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the CHRNB3 gene. For example, the recognition sequence can be located about 10, about 20, about 30, about 40, about 50, about 100, about 200, about 300, about 400, about 500, or about 1,000 nucleotides from the start codon.

As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify a CHRNB3 genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of CHRNB3 nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in a CHRNB3 genomic nucleic acid molecule, or it can be a nickase that creates a single-strand break in a CHRNB3 genomic nucleic acid molecule. Additional examples of Cas proteins include, but are not limited to, Cas1, Cas1B, Cas2, Cas3, Cas4, Cas5, Cas5e (CasD), Cas6, Cas7, Cas6f, Cas7, Cas8a1, Cas8a2, Cas8b, Cas8c, Cas9 (Csn1 or Csx12), Cas10, Cas10d, Cas12a, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. In some embodiments, a Cas system, such as Cas12a, can have multiple gRNAs encoded into a single crRNA. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of CHRNB3 genomic nucleic acid molecules can be generated by contacting a cell with a Cas protein and one or more gRNAs that hybridize to one or more gRNA recognition sequences within a target genomic locus in the CHRNB3 genomic nucleic acid molecule. The gRNA recognition sequence can include or be proximate to the start codon of a CHRNB3 genomic nucleic acid molecule or the stop codon of a CHRNB3 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in a CHRNB3 genomic nucleic acid molecule can be located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-3′, where N is any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within a CHRNB3 genomic nucleic acid molecule. An exemplary gRNA is a gRNA effective to direct a Cas enzyme to bind to or cleave a CHRNB3 genomic nucleic acid molecule, wherein the gRNA comprises a DNA-targeting segment that hybridizes to a gRNA recognition sequence within the CHRNB3 genomic nucleic acid molecule. Exemplary gRNAs comprise a DNA-targeting segment that hybridizes to a gRNA recognition sequence present within a CHRNB3 genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

The Cas protein and the gRNA form a complex, and the Cas protein cleaves the target CHRNB3 genomic nucleic acid molecule. The Cas protein can cleave the nucleic acid molecule at a site within or outside of the nucleic acid sequence present in the target CHRNB3, genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the CHRNB3 genomic nucleic acid molecule to which a DNA-targeting segment of a gRNA will bind.

Such methods can result, for example, in a CHRNB3 genomic nucleic acid molecule in which a region of the gene is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the CHRNB3 genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In some embodiments, the CHRNB3 inhibitor is a small molecule. In some embodiments, the CHRNB3 inhibitor is an antibody. In some embodiments, the CHRNB3 inhibitor comprises an inhibitory nucleic acid molecule, such as, for example an antisense nucleic acid molecule, an siRNA, or an shRNA.

In some embodiments, the dose of the CHRNB3 inhibitor can be reduced by about 10%, by about 20%, by about 30%, by about 40%, by about 50%, by about 60%, by about 70%, by about 80%, or by about 90% for subjects that are heterozygous for one or more CHRNB3 variant nucleic acid molecules compared to subjects that are CHRNB3 reference (who may receive a standard dosage amount). In some embodiments, the dose of the CHRNB3 inhibitor can be reduced by about 10%, by about 20%, by about 30%, by about 40%, or by about 50%. In addition, the dose of the CHRNB3 inhibitor in subjects that are heterozygous for one or more CHRNB3 variant nucleic acid molecules can be administered less frequently compared to subjects that are CHRNB3 reference. The dose can also be changed based on BMI, age, sex, etc.

In some embodiments, the methods further comprise detecting the presence or absence of a CHRNB3 variant nucleic acid molecule and/or a CHRNB3 predicted loss-of-function polypeptide in a biological sample from the subject. In some embodiments, the CHRNB3 variant nucleic acid molecule is a genomic nucleic acid molecule. In some embodiments, the CHRNB3 variant nucleic acid molecule is an mRNA molecule. In some embodiments, the CHRNB3 variant nucleic acid molecule is a cDNA molecule produced from an mRNA molecule. In some embodiments, the CHRNB3 variant nucleic acid molecule is a missense variant, a splice-site variant, a stop-gain variant, a start-loss variant, a stop-loss variant, a frameshift variant, or an in-frame indel variant, or a variant that encodes a truncated or mutated CHRNB3 polypeptide.

Detecting the presence or absence of a CHRNB3 variant nucleic acid molecule in a biological sample from a subject and/or determining whether a subject has a CHRNB3 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, the detecting step comprises obtaining or having obtained a biological sample from the subject, and performing or having performed an assay on the biological sample to determine whether the subject has the CHRNB3 variant nucleic acid molecule, and/or a CHRNB3 predicted loss-of-function polypeptide. In some embodiments, the assay is a sequence analysis that comprises sequencing at least a portion of the nucleotide sequence of the CHRNB3 genomic nucleic acid molecule in the biological sample. In some embodiments, the assay is a sequence analysis that comprises sequencing at least a portion of the nucleotide sequence of the CHRNB3 mRNA molecule in the biological sample. In some embodiments, the assay is a sequence analysis that comprises sequencing at least a portion of the nucleotide sequence of the CHRNB3 cDNA molecule produced from an mRNA molecule in the biological sample.

In some embodiments, the sequence analysis comprises: a) contacting the biological sample with a primer hybridizing to a portion of the nucleotide sequence of the CHRNB3 nucleic acid molecule that is proximate to a CHRNB3 variant nucleic acid molecule position; b) extending the primer at least through the CHRNB3 variant nucleic acid molecule position; and c) determining whether the extension product of the primer comprises a variant nucleotide at the CHRNB3 variant nucleic acid molecule position. In some embodiments, the sequence analysis comprises sequencing the entire nucleic acid molecule in the biological sample.

In some embodiments, the assay is a sequence analysis that comprises: a) amplifying at least a portion of the CHRNB3 nucleic acid molecule in the biological sample, wherein the portion comprises a CHRNB3 variant nucleic acid molecule position; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the CHRNB3 variant nucleic acid molecule position; and d) detecting the detectable label. In some embodiments, the CHRNB3 nucleic acid molecule in the biological sample is mRNA and the mRNA is reverse transcribed into cDNA prior to the amplifying step.

In some embodiments, the assay is a sequence analysis that comprises contacting the CHRNB3 nucleic acid molecule in the biological sample with an alteration-specific probe comprising a detectable label, wherein the alteration-specific probe comprises a nucleotide sequence which hybridizes under stringent conditions to a CHRNB3 variant nucleic acid molecule position and detecting the detectable label.

In some embodiments, the assay is an immunoassay for detecting the presence of a CHRNB3 predicted loss-of-function polypeptide. In some embodiments, mass spectrometry is used for detecting the presence of a CHRNB3 predicted loss-of-function polypeptide.

In some embodiments, the methods further comprise determining the subject's gene burden of having a CHRNB3 variant nucleic acid molecule and/or a CHRNB3 predicted loss-of-function polypeptide. When the subject has a lower gene burden, the subject is administered or continued to be administered the CHRNB3 inhibitor in a standard dosage amount. When the subject has a greater gene burden, the subject is administered or continued to be administered the CHRNB3 inhibitor in an amount that is the same as or less than the standard dosage amount. In some embodiments, the subject's gene burden represents a weighted sum of a plurality of genetic variants associated with protection against developing a smoking addiction/disorder. In some embodiments, the gene burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, or at least about 1,000 genetic variants associated with smoking addiction/disorder. In some embodiments, the gene burden may be divided into quintiles, e.g., top quintile, intermediate quintile, and bottom quintile, wherein the top quintile of gene burden corresponds to the lowest risk group and the bottom quintile of gene burden corresponds to the highest risk group.

The sequence analysis to determine whether a subject has a CHRNB3 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In any of the embodiments described herein, when the subject is CHRNB3 reference, the subject can be administered the CHRNB3 inhibitor in a standard dosage amount. When the subject is heterozygous for a CHRNB3 variant nucleic acid molecule encoding a CHRNB3 predicted loss-of-function polypeptide, the subject can be administered the CHRNB3 inhibitor in a dosage amount that is the same as or less than a standard dosage amount.

In some embodiments, when the subject is CHRNB3 reference, the subject is also administered a therapeutic agent that treats or inhibits smoking addiction/disorder in a standard dosage amount. In some embodiments, when the subject is heterozygous for a CHRNB3 variant nucleic acid molecule, the subject is also administered a therapeutic agent that treats or inhibits smoking addiction/disorder in a dosage amount that is the same as or less than a standard dosage amount.

In some embodiments, the treatment methods further comprise detecting the presence or absence of a CHRNB3 predicted loss-of-function polypeptide in a biological sample from the subject. In some embodiments, when the subject does not have a CHRNB3 predicted loss-of-function or polypeptide, the subject is also administered a therapeutic agent that treats or inhibits smoking addiction/disorder in a standard dosage amount. In some embodiments, when the subject has a CHRNB3 predicted loss-of-function polypeptide, the subject is also administered a therapeutic agent that treats or inhibits smoking addiction/disorder in a dosage amount that is the same as or less than a standard dosage amount.

The present disclosure also provides methods of treating a subject with a CHRNB3 inhibitor, wherein the subject has a smoking addiction/disorder or is at risk of developing a smoking addiction/disorder. The methods comprise determining whether the subject has a CHRNB3 variant nucleic acid molecule by obtaining or having obtained a biological sample from the subject, and performing or having performed a sequence analysis on the biological sample to determine if the subject has a genotype comprising the CHRNB3 variant nucleic acid molecule. When the subject is CHRNB3 reference, the method further comprises administering or continuing to administer to the subject CHRNB3 inhibitor in a standard dosage amount. When the subject is heterozygous for the CHRNB3 variant nucleic acid molecule, the method further comprises administering or continuing to administer to the subject the CHRNB3 inhibitor in a dosage amount that is the same as or less than a standard dosage amount. The presence of a genotype having the CHRNB3 variant nucleic acid molecule indicates the subject has a decreased risk of developing the smoking addiction/disorder or has a decreased risk of developing a more severe form of the smoking addiction/disorder. Determining whether the subject has a genotype comprising the CHRNB3 variant nucleic acid molecule can be carried out as described herein.

In some embodiments, the subject CHRNB3 reference, and the subject is administered or continued to be administered the CHRNB3 inhibitor in a standard dosage amount. In some embodiments, the subject is heterozygous for the CHRNB3 variant nucleic acid molecule, and the subject is administered or continued to be administered the CHRNB3 inhibitor in a dosage amount that is the same as or less than a standard dosage amount.

In any of the embodiments described herein, the method can further comprise administering a therapeutic agent for treating a smoking addiction/disorder to the subject.

Administration of the CHRNB3 inhibitor and/or other therapeutic agents that treat or inhibit a smoking addiction/disorder can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the CHRNB3 inhibitor and/or other therapeutic agents that treat or inhibit a smoking addiction/disorder can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

In any of the embodiments described herein, the subject can have or be at risk of developing any one or more of smoking addiction/disorders described herein or known in the art. In some embodiments, the subject is CHRNB3 reference. In some embodiments, the subject is heterozygous for a CHRNB3 variant nucleic acid molecule. In some embodiments, the subject is homozygous for a CHRNB3 variant nucleic acid molecule.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease and/or reduction in a smoking addiction/disorder, a decrease and/or reduction in the severity of a smoking addiction/disorder, a decrease and/or reduction in symptoms and smoking addiction/disorder-related effects, delaying the onset of symptoms and smoking addiction/disorder-related effects, reducing the severity of symptoms of smoking addiction/disorder-related effects, reducing the number of symptoms and smoking addiction/disorder-related effects, reducing the latency of symptoms and smoking addiction/disorder-related effects, an amelioration of symptoms and smoking addiction/disorder-related effects, reducing secondary symptoms, reducing secondary infections, preventing relapse to a smoking addiction/disorder, decreasing the number or frequency of relapse episodes, increasing latency between symptomatic episodes, increasing time to sustained progression, speeding recovery, or increasing efficacy of or decreasing resistance to alternative therapeutics, and/or an increased survival time of the affected subject/host animal, following administration of the agent or composition comprising the agent. A prophylactic effect may comprise a complete or partial avoidance and/or inhibition or a delay of smoking addiction/disorder development and/or progression (such as, for example, a complete or partial avoidance/inhibition or a delay), and an increased survival time of the affected subject/host animal, following administration of a therapeutic protocol. Treatment of smoking addiction/disorder encompasses the treatment of subjects already diagnosed as having any form of smoking addiction/disorder at any clinical stage or manifestation, the delay of the onset or evolution or aggravation or deterioration of the symptoms or signs of smoking addiction/disorder, and/or preventing and/or reducing the severity of a smoking addiction/disorder.

The present disclosure also provides methods of identifying a subject having an increased risk of developing a smoking addiction/disorder. In some embodiments, the methods comprise determining or having determined in a biological sample obtained from the subject the presence or absence of a CHRNB3 variant nucleic acid molecule (such as a genomic nucleic acid molecule, mRNA molecule, and/or cDNA molecule). When the subject lacks a CHRNB3 variant nucleic acid molecule (i.e., the subject is genotypically categorized as a CHRNB3 reference), then the subject has an increased risk of developing a smoking addiction/disorder. When the subject has a CHRNB3 variant nucleic acid molecule (i.e., the subject is heterozygous or homozygous for a CHRNB3 variant nucleic acid molecule), then the subject has a decreased risk of developing a smoking addiction/disorder compared to a subject who is CHRNB3 reference.

Having a single copy of a CHRNB3 variant nucleic acid molecule is more protective of a subject from developing a smoking addiction/disorder than having no copies of a CHRNB3 variant nucleic acid molecule. Without intending to be limited to any particular theory or mechanism of action, it is believed that a single copy of a CHRNB3 variant nucleic acid molecule (i.e., heterozygous for a CHRNB3 variant nucleic acid molecule) is protective of a subject from developing a smoking addiction/disorder, and it is also believed that having two copies of a CHRNB3 variant nucleic acid molecule (i.e., homozygous for a CHRNB3 variant nucleic acid molecule) may be more protective of a subject from developing a smoking addiction/disorder, relative to a subject with a single copy. Thus, in some embodiments, a single copy of a CHRNB3 variant nucleic acid molecule may not be completely protective, but instead, may be partially or incompletely protective of a subject from developing a smoking addiction/disorder. While not desiring to be bound by any particular theory, there may be additional factors or molecules involved in the development of a smoking addiction/disorder that are still present in a subject having a single copy of a CHRNB3 variant nucleic acid molecule, thus resulting in less than complete protection from the development of a smoking addiction/disorder.

Determining whether a subject has a CHRNB3 variant nucleic acid molecule in a biological sample from a subject and/or determining whether a subject has a CHRNB3 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

The present disclosure also provides methods of identifying a subject having an increased risk of developing a smoking addiction/disorder, wherein the methods comprise determining or having determined the subject's gene burden of having one or more CHRNB3 variant genomic nucleic acid molecules described herein, one or more CHRNB3 variant mRNA molecules described herein, or one or more variant cDNA molecules described herein, and/or one or more CHRNB3 predicted loss-of-function polypeptides or missense polypeptides described herein. The greater the gene burden the subject has, the lower the risk of developing a smoking addiction/disorder. The lower the gene burden the subject has, the greater the risk of developing a smoking addiction/disorder.

In some embodiments, the subject's gene burden of having a plurality of (or all) CHRNB3 variant nucleic acid molecules represents a weighted sum of a plurality of genetic variants associated with protection against developing a smoking addiction/disorder. In some embodiments, the gene burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, or at least about 1,000 genetic variants associated with smoking addiction/disorder. In some embodiments, when the subject has a gene burden greater than a threshold score, the subject has a decreased risk of developing a smoking addiction/disorder. In some embodiments, when the subject has a gene burden below a threshold score, the subject has an increased risk of developing a smoking addiction/disorder.

In some embodiments, the gene burden may be divided into quintiles, e.g., top quintile, intermediate quintile, and bottom quintile, wherein the top quintile of gene burden corresponds to the lowest risk group and the bottom quintile of gene burden corresponds to the highest risk group.

In some embodiments, when a subject is identified as having an increased risk of developing a smoking addiction/disorder, the subject is further treated with a therapeutic agent that treats or inhibits smoking addiction/disorder and/or a CHRNB3 inhibitor, as described herein. For example, when the subject is CHRNB3 reference, and therefore has an increased risk of developing a smoking addiction/disorder, the subject can be administered a CHRNB3 inhibitor in a standard dosage amount. In some embodiments, such a subject is also administered a therapeutic agent that treats or inhibits a smoking addiction/disorder. In some embodiments, when the subject is heterozygous for a CHRNB3 variant nucleic acid molecule, the subject is administered the CHRNB3 inhibitor in a dosage amount that is the same as or less than a standard dosage amount and can also be administered a therapeutic agent that treats or inhibits a smoking addiction/disorder. In some embodiments, the subject is CHRNB3 reference. In some embodiments, the subject is heterozygous for a CHRNB3 variant nucleic acid molecule.

The present disclosure also provides, in any of the methods described herein, the detection or determination of the presence of a CHRNB3 variant genomic nucleic acid molecule, a CHRNB3 variant mRNA molecule, and/or a CHRNB3 variant cDNA molecule produced from an mRNA molecule in a biological sample from a subject. It is understood that gene sequences within a population and mRNA molecules encoded by such genes can vary due to polymorphisms such as single-nucleotide polymorphisms. The sequences provided herein for the CHRNB3 variant nucleic acid molecules disclosed herein are only exemplary sequences. Other sequences for the CHRNB3 variant nucleic acid molecules are also possible.

The biological sample can be derived from any cell, tissue, or biological fluid from the subject. The sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The sample used in the methods disclosed herein will vary based on the assay format, nature of the detection method, and the tissues, cells, or extracts that are used as the sample. A biological sample can be processed differently depending on the assay being employed. For example, when detecting any of the nucleic acid molecules, preliminary processing designed to isolate or enrich the sample for the genomic DNA can be employed. A variety of known techniques may be used for this purpose. When detecting the presence of or level of any CHRNB3 mRNA molecule, different techniques can be used enrich the biological sample with mRNA. Various methods to detect the presence or level of a mRNA or the presence of a particular variant genomic DNA locus can be used.

In some embodiments, detecting a CHRNB3 variant nucleic acid molecule in a subject comprises assaying or performing a sequence analysis on a biological sample obtained from the subject to determine whether a CHRNB3 genomic nucleic acid molecule, a CHRNB3 mRNA molecule, or a CHRNB3 cDNA molecule produced from an mRNA molecule in the biological sample is a CHRNB3 variant nucleic acid molecule.

In some embodiments, the methods of detecting the presence or absence of a CHRNB3 variant nucleic acid molecule (such as, for example, a genomic nucleic acid molecule, an mRNA molecule, and/or a cDNA molecule) in a subject, comprise performing an assay on a biological sample obtained from the subject, which assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising a CHRNB3 genomic nucleic acid molecule or mRNA molecule, and if mRNA, optionally reverse transcribing the mRNA into cDNA. Such assays can comprise, for example determining the identity of these positions of the particular, CHRNB3 nucleic acid molecule. In some embodiments, the method is an in vitro method.

In some embodiments, the determining step, detecting step, or sequence analysis comprises sequencing at least a portion of the nucleotide sequence of the CHRNB3 genomic nucleic acid molecule, the CHRNB3 mRNA molecule, or the CHRNB3 cDNA molecule produced from the mRNA molecule in the biological sample, wherein the sequenced portion comprises one or more variations that cause a loss-of-function or missense (partial or complete) or are predicted to cause a loss-of-function or missense (partial or complete), such CHRNB3 nucleic acid molecules described herein.

In any of the methods described herein, the determining step, detecting step, or sequence analysis comprises sequencing at least a portion of the nucleotide sequence of the CHRNB3 nucleic acid molecule in the biological sample, wherein the sequenced portion comprises a position corresponding to a variant nucleic acid molecule position, wherein when a variant nucleotide at the variant nucleic acid molecule position is detected, the CHRNB3 nucleic acid molecule in the biological sample is a CHRNB3 variant nucleic acid molecule.

In some embodiments, the determining step, detecting step, or sequence analysis comprises: a) contacting the biological sample with a primer hybridizing to a portion of the nucleotide sequence of the CHRNB3 nucleic acid molecule that is proximate to a variant nucleic acid molecule position; b) extending the primer at least through the variant nucleic acid molecule position; and c) determining whether the extension product of the primer comprises a variant nucleotide at the variant nucleic acid molecule position.

In some embodiments, the assay comprises sequencing the entire nucleic acid molecule. In some embodiments, only a CHRNB3 genomic nucleic acid molecule is analyzed. In some embodiments, only a CHRNB3 mRNA is analyzed. In some embodiments, only a CHRNB3 cDNA obtained from CHRNB3 mRNA is analyzed.

In some embodiments, the determining step, detecting step, or sequence analysis comprises: a) amplifying at least a portion of the CHRNB3 nucleic acid molecule that encodes the CHRNB3 polypeptide, wherein the portion comprises a variant nucleic acid molecule position; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the variant nucleic acid molecule position; and d) detecting the detectable label.

The alteration-specific probes or alteration-specific primers described herein comprise a nucleic acid sequence which is complementary to and/or hybridizes, or specifically hybridizes, to a CHRNB3 variant nucleic acid molecule, or the complement thereof. In some embodiments, the alteration-specific probes or alteration-specific primers comprise or consist of at least about 1, at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, or at least about 50 nucleotides. In some embodiments, the alteration-specific probes or alteration-specific primers comprise or consist of at least 15 nucleotides. In some embodiments, the alteration-specific probes or alteration-specific primers comprise or consist of at least 15 nucleotides to at least about 35 nucleotides. In some embodiments, alteration-specific probes or alteration-specific primers hybridize to CHRNB3 variant genomic nucleic acid molecules, CHRNB3 variant mRNA molecules, and/or CHRNB3 variant cDNA molecules under stringent conditions.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse transcribed into a cDNA prior to the amplifying step. In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to a CHRNB3 variant genomic sequence, variant mRNA sequence, or variant cDNA sequence and not the corresponding CHRNB3 reference sequence under stringent conditions and determining whether hybridization has occurred.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising a CHRNB3 variant genomic nucleic acid molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence-based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na+ ion, typically about 0.01 to 1.0 M Na+ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, detecting the presence of a predicted loss-of-function polypeptide or missense polypeptide comprises performing an assay on a sample obtained from a subject to determine whether a CHRNB3 polypeptide in the subject contains one or more variations that causes the polypeptide to have a loss-of-function (partial or complete) or predicted loss-of-function (partial or complete) or missense variant. In some embodiments, the assay comprises sequencing at least a portion of the CHRNB3 polypeptide that comprises a variant position. In some embodiments, the detecting step comprises sequencing the entire polypeptide. Identification of a variant amino acid at the variant position of the CHRNB3 polypeptide indicates that the CHRNB3 polypeptide is a predicted loss-of-function or missense CHRNB3 polypeptide. In some embodiments, the assay comprises an immunoassay for detecting the presence of a polypeptide that comprises a variant. Detection of a variant amino acid at the variant position of the CHRNB3 polypeptide indicates that the CHRNB3 polypeptide is a CHRNB3 predicted loss-of-function or missense polypeptide.

In some embodiments, isolated nucleic acid molecules hybridize to CHRNB3 variant nucleic acid molecules, nucleic acid molecules encoding a CHRNB3 polypeptide, or nucleic acid molecules encoding a reference CHRNB3 polypeptide or a functional CHRNB3 polypeptide (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to CHRNB3 variant genomic nucleic acid molecules, CHRNB3 variant mRNA molecules, and/or CHRNB3 variant cDNA molecules.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to PNPLA3 genomic nucleic acid molecules encoding a CHRNB3 polypeptide, CHRNB3 mRNA molecules, and/or CHRNB3 cDNA molecules encoding a CHRNB3 polypeptide.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can also be used to detect a nucleotide variation within any of the CHRNB3 variant genomic nucleic acid molecules, CHRNB3 variant mRNA molecules, and/or CHRNB3 variant cDNA molecules disclosed herein. The primers described herein can be used to amplify CHRNB3 variant genomic nucleic acid molecules, CHRNB3 variant mRNA molecules, or CHRNB3 variant cDNA molecules, or a fragment thereof.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following example is provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### Example

Deleterious Variants in CHRNB3 Confer Protection from Heavy Smoking.
An Exome-Wide Association Study of Heaviness of Smoking (Number of Cigarettes Smoked per day) was performed in 37,897 Mexican Americans from the Mexican City Prospective Study (MCPS) cohort. The strongest association observed in this analysis was with CHRNB3, where a missense variant (Glu284Gly), enriched in Mexican Americans (but absent elsewhere in the world), was associated with smoking fewer cigarettes per day (protective association). Heterozygous carriers (A/G) of this variant smoked on average 1.2 fewer cigarettes per day compared to wild types (A/A), and homozygous carriers (G/G) of this variant smoked on average 4.5 fewer cigarettes per day compared to wild types (AA). Further analysis of British Europeans from the UK Biobank revealed a nominally significant protective association between loss of function variants in CHRNB3 and number of cigarettes smoked per day. Heterozygous carriers of loss of function variants in CHRNB3 smoked on average 3 fewer cigarettes per day compared to non-carriers. CHRNB3 encodes a beta-3 subunit of nicotine receptors expressed densely in the human midbrain. The findings overall suggest that loss of CHRNB3 is protective against heavy smoking. Hence, therapeutic inhibition of CHRNB3 in the brain can be used to treat smoking addiction.

### Exome-Wide Association Study in Mexican Americans

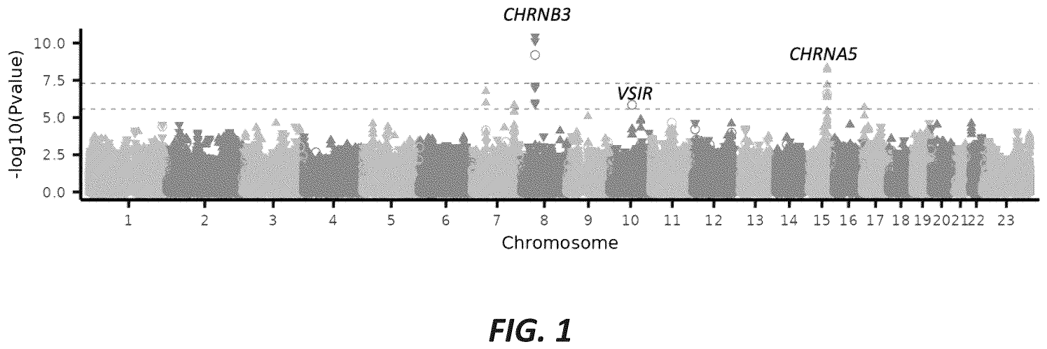

The associations of all rare coding variants were studied in the human genome aggregated per gene based on specific classes such as predicted loss of function (pLoF), deleterious missense variants, etc. with the number of cigarettes smoked per day (cig-per-day) in 37,897 individuals of Mexican American ancestry from the MCPS cohort. The results are shown in the Manhattan plot in FIG. 1.

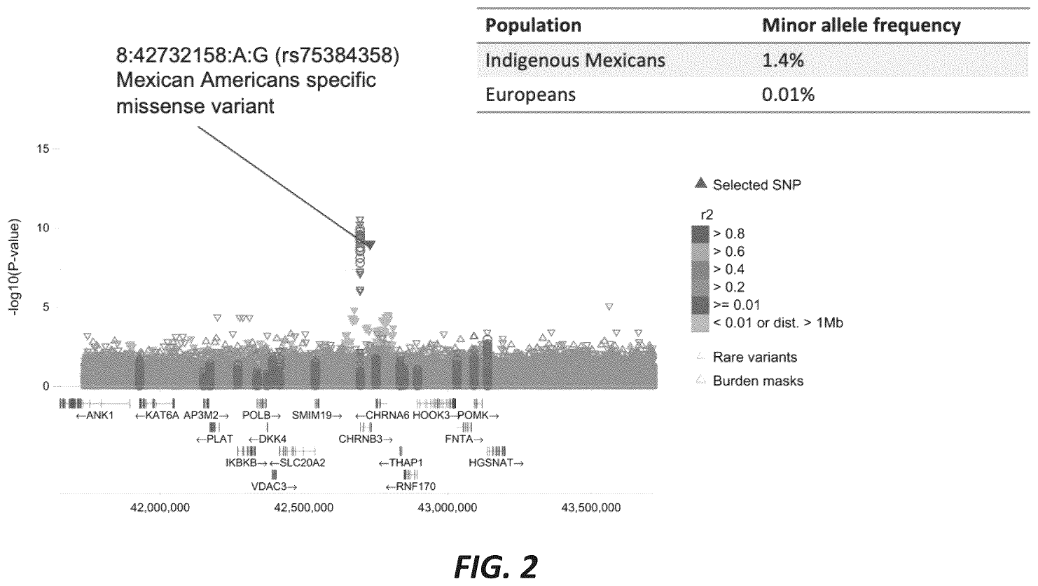

The top association was observed for CHRNB3 in chromosome 8. This association is primarily mediated by a single missense variant (Glu284Gly) in CHRNB3. The missense variant is characterized by the amino acid change from glutamic acid to glycine at position 284 in the CHRNB3 protein. This substitution is predicted as deleterious by five different prediction algorithms. The minor allele frequency of this missense variant is 1.4% in Indigenous Mexicans as compared to a mere 0.01% in European ancestries suggesting that this variant is specific to Mexican American (particularly Indigenous Americans) ancestry. The plot in FIG. 2 shows the association of rare variants in the genomic region around CHRNB3 in chromosome 8. The missense variant is labeled.

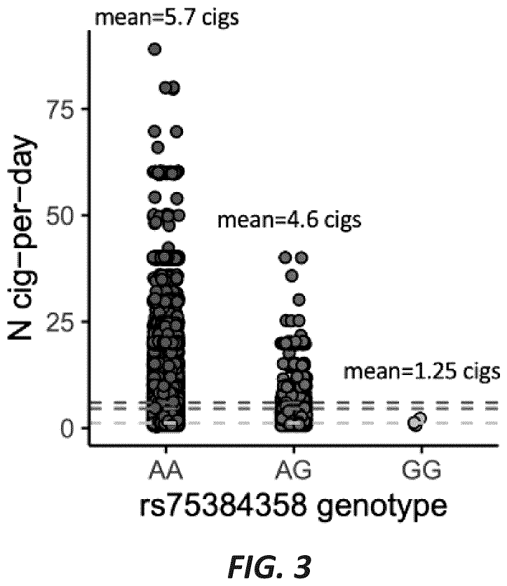

The plot in FIG. 3 shows the distribution of cig-per-day in the three genotype groups of the Glu284Gly missense variant. Wild-type carriers (A/A) smoked on average 5.7 cigs per day, heterozygous carriers (A/G) smoked on average 4.6 cigs per day and homozygous carriers on average smoked 1.25 cigs per day. The mean difference between homozygous and wild-type carriers is 4.45 cigs per day which is much higher than the mean difference between heterozygous and wild-type carriers (1.1 cigs per day), suggesting that CHRNB3 is fully inactive in homozygous carriers.

The table below shows the number of carriers of wild-type, heterozygous, and homozygous genotypes, the association effect size in standard deviations (based on linear regression; 1 SD is 5.75 cigs per day), 95% confidence interval, and P value.

| | | | Wild | | | | | |
|---|---|---|---|---|---|---|---|---|
| Variant/ | Reference | Alternative | type | Heterozygous | Homozygous | | 95% | P |
| Burden | allele | allele | (A/A) | (A/G) | (G/G) | Beta | CI | value |
| rs75384358 | A | G | 37,206 | 687 | 4 | −0.20 | −0.26, | 1.1e−9 |
| | | | | | | SD | −0.13 | |

Association of CHRNB3 with Cig Per Day in British Europeans

Motivated by the primary discovery based on Mexican Americans, we studied the association of rare variants in CHRNB3 with cig per day in ˜100,000 British Europeans from the UK Biobank. Although the missense variant Glu284Gly is extremely rare in Europeans, the relatively larger sample size of UK Biobank compared to MCPS enabled us to look for nominal associations with rare pLOFs in CHRNB3. As expected, we observed a nominally significant protective association between rare pLOFs in CHRNB3 and cig per day. Heterozygous carriers of rare (minor allele frequency <1%) pLOFs in CHRNB3 (n=79 carriers) on average smoked around 3 fewer cigarettes per day than non-carriers in the UK Biobank. The association statistics are shown in the table below. This association complements our primary discovery and increases our confidence in the direction of the association. That is, the findings suggest that loss of CHRNB3 is protective against heavy smoking.

| | | | Wild | | | | | |
|---|---|---|---|---|---|---|---|---|
| Variant/ | Reference | Alternative | type | Heterozygous | Homozygous | | 95% | P |
| Burden | allele | allele | (A/A) | (A/G) | (G/G) | Beta | CI | value |
| pLOFs with | — | — | 102,242 | 79 | 0 | −0.27 | −0.48, | 0.008 |
| MAF <1% | | | | | | SD | −0.07 | |

Association of Common Variants in CHRNB3 with Cig Per Day in Mexican Americans and British Europeans

The association of common variants in and around CHRNB3 with cig per day is already known and reported in the literature since 2010 (Thorgeirsson et al. Nat Gen 2010; https://pubmed.ncbi.nlm.nih.gov/20418888/). We observe the same in our sample. The table below summarizes the association of a common (MAF=20% in Mexican Americans and 30% in Europeans) noncoding variant near CHRNB3 with cig per day in the MCPS and UK Biobank cohorts.

| | | | | Wild | Hetero- | Homo- | | | |
|---|---|---|---|---|---|---|---|---|---|
| | Variant/ | Reference | Alternative | type | zygous | zygous | | 95% | P |
| Cohort | Burden | allele | allele | (A/A) | (A/G) | (G/G) | Beta | CI | value |
| MCPS | rs6984252 | C | G | 17,977 | 16,281 | 3,639 | −0.03 | −0.04, | 5.2e−5 |
| | | | | | | | | −0.01 | |
| UKB | rs6984252 | C | G | 64,947 | 34,026 | 4,350 | −0.04 | −0.05, | 2.1e−12 |
| | | | | | | | | −0.03 | |

Although the common variant genetic associations were known, they do not directly implicate the gene driving the association. For example, there are two nicotinic receptor genes located in the GWAS signal in chromosome 8 namely CHRNB3 and CHRNA6. It is difficult to tell which gene is driving the association. Also, the common variants do not inform on the direction of the association. On the other hand, our rare variant discovery directly pinpoints the driver gene and informs on the direction of the association (in our case, the loss of function of CHRNB3 protects against heavy smoking).

Association of CHRNB3 with Cig Per Day in East Asian Populations

The associations of CHRNB3 coding variants with cig per day were studied in the East Asian populations by analyzing genetic results from Japan Biobank (https://pheweb.jp/variant/8:42552742-G-A). In the analysis, a specific pLOF (splice donor variant c.52+1G>A, rs147306385) was found that associated strongly with cig per day in the protective direction. This variant was enriched in the East Asian populations with a minor allele frequency of 0.5% but extremely rare in other populations (MAF<0.01%). The effect size of the EAS-specific pLOF was similar to that of AMR-enriched Glu284Gly. The finding further validated the protective association of CHRNB3 with cig per day.

| | | | | Wild | Hetero- | Homo- | | | |
|---|---|---|---|---|---|---|---|---|---|
| | Variant/ | Reference | Alternative | type | zygous | zygous | | 95% | P |
| Cohort | Burden | allele | allele | (A/A) | (A/G) | (G/G) | Beta | CI | value |
| MCPS | Glu284Gly | A | G | 37,206 | 687 | 4 | −0.19 | −0.13, | 1.1e−9 |
| | (rs75384358)* | | | | | | | −0.26 | |
| Japan | Splice donor | G | A | 74,175 | 716 | 2 | −0.19 | −0.12, | 3.9e−8 |
| Biobank | (rs147306385) | | | | | | | −0.26 | |

### Biology of CHRNB3 and the Novel Therapeutic Opportunity

CHRNB3 encodes the b3 subunit of nicotinic acetylcholine receptors expressed densely in the human midbrain. Nicotinic receptors mediate the rewarding and aversive effects of nicotine. Rewarding effects are positive effects experienced by the smoker (elevated mood, relaxed feeling, increased focus, etc.) and aversive effects are negative effects experienced by the smoker, which typically occur when the level of nicotine rises beyond a threshold in the brain (examples: restlessness, anxiety, tremors, tingling sensation in the skin, etc.).

Existing drugs for smoking cessation act primarily by blocking the rewarding effects of smoking. For example, varenicline (from Pfizer) is a partial agonist/antagonist of b2 subunit (encoded by CHRNB2). This discovery suggests that CHRNB3 might primarily regulate the aversive effects of smoking. Hence, therapeutic inhibition of CHRNB3 in the brain will increase the sensitivity of patients to nicotine-aversive effects thereby preventing them from smoking more cigarettes. Notably, this is a different mechanism compared to varenicline's mechanism of action. Hence, CHRNB3 inhibitors could in principle complement varenicline in the treatment of smoking addiction or could serve as an alternative drug to varenicline.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A method to reduce smoking in a subject, the method comprising administering an inhibitor of cholinergic receptor nicotinic beta 3 subunit (CHRNB3) to the subject.

2. The method of claim 1, wherein the subject smokes 1-2 fewer cigarettes per day as compared to smoking prior to treatment.

3. The method of claim 1, wherein the subject smokes 3-4 fewer cigarettes per day as compared to smoking prior to treatment.

4. The method of claim 1, wherein the subject smokes 4-5 fewer cigarettes per day as compared to smoking prior to treatment.

5. The method of claim 1, wherein the subject smokes 4-6 fewer cigarettes per day as compared to smoking prior to treatment.

6. The method of claim 1, wherein the subject smokes 7 or more fewer cigarettes per day as compared to smoking prior to treatment.

7. The method of claim 1, wherein the inhibitor of CHRNB3 comprises a small molecule.

8. The method of claim 1, wherein the inhibitor or CHRNB3 comprises an inhibitory nucleic acid molecule.

9. The method of claim 8, wherein the inhibitory nucleic acid molecule comprises an antisense nucleic acid molecule, a small interfering RNA (siRNA), or a short hairpin RNA (shRNA) that hybridizes to a CHRNB3 mRNA.

10. The method of claim 1, wherein the inhibitor of CHRNB3 comprises a Cas protein and guide RNA (gRNA) that hybridizes to a gRNA recognition sequence within a CHRNB3 genomic nucleic acid molecule.

11. The method of claim 1, further comprising detecting the presence or absence of an CHRNB3 variant nucleic acid molecule and/or a CHRNB3 predicted loss-of-function polypeptide or missense polypeptide in a biological sample from the subject.

12. The method of claim 11, wherein the detecting step is carried out in vitro.

13. The method of claim 12, wherein the detecting step comprises:
obtaining or having obtained a biological sample from the subject; and
performing or having performed an assay on the biological sample to determine whether the subject has the CHRNB3 variant nucleic acid molecule, and/or a CHRNB3 predicted loss-of-function polypeptide or missense polypeptide.

14. The method of claim 11, wherein the subject has an absence of an CHRNB3 variant nucleic acid molecule and/or a CHRNB3 predicted loss-of-function polypeptide and is administered the CHRNB3 inhibitor.

15. The method of claim 11, wherein the subject is heterozygous for a CHRNB3 variant nucleic acid molecule, and the subject is administered the CHRNB3 inhibitor.

16. A method of treating a subject with a cholinergic receptor nicotinic beta 3 subunit (CHRNB3) inhibitor, wherein the subject is need of reducing smoking or at risk of increasing smoking, the method comprising the steps of:
determining whether the subject has a CHRNB3 variant nucleic acid molecule by:
obtaining or having obtained a biological sample from the subject; and
performing or having performed a sequence analysis on the biological sample to determine if the subject has a genotype comprising the CHRNB3 variant nucleic acid molecule; and
administering or continuing to administer the CHRNB3 inhibitor in a standard dosage amount to a CHRNB3 reference subject; and
administering or continuing to administer the CHRNB3 inhibitor in a dosage amount that is the same as or less than a standard dosage amount to a subject that is heterozygous for the CHRNB3 variant nucleic acid molecule;
wherein the presence of a genotype having the CHRNB3 variant nucleic acid molecule indicates the subject has a decreased risk of increase in smoking/addiction.
